(19) United States
(12) Patent Application Publication
(10) Pub. No.: US 2015/0126964 A1
(43) Pub. Date: May 7, 2015

(54) DRUG DELIVERY ACROSS THE BLOOD-BRAIN BARRIER USING MAGNETICALLY HEATABLE ENTITIES

(71) Applicant: CORPORATION DE L'ECOLE POLYTECHNIQUE DE MONTREAL, Montreal (CA)

(72) Inventors: Sylvain Martel, Quebec (CA); Seyed Nasrollah Tabatabaei Shafie, Montreal (CA)

(21) Appl. No.: 14/400,349

(22) PCT Filed: May 13, 2013
(86) PCT No.: PCT/CA2013/050368

§ 371 Date: Nov. 11, 2014

Related U.S. Application Data
(60) Provisional application No. 61/646,874, filed on May 14, 2012.
(60) Provisional application No. 61/646,357, filed on May 13, 2012.

(51) Int. Cl. A61K 47/46; A61K 41/00; A61F 7/12

(52) U.S. Cl. CPC A61K 47/46; A61F 7/12; A61K 41/0052; A61F 2007/0002
USPC 604/507; 604/113; 514/772

(57) ABSTRACT

It has been discovered that compositions comprising magnetically heatable entities (MHEs), therapeutic agents and optional carriers such as hydrogels can be piloted from an injection point in a blood vessel to a specific location of the blood-brain barrier (BBB) using for example, a magnetic resonance imaging (MRI) device for propelling, steering and tracking of MHEs. Once the MHEs have reached their target location at or near the desired blood vessel of the BBB, an alternating magnetic field causes the MHEs to controllably heat up, thereby reversibly increasing the permeability of the BBB and allowing the therapeutic (or cytotoxic) agent to enter brain tissue.

### TECHNICAL FIELD

This invention relates generally to drug delivery to brain tissue. More specifically, this invention relates to a system, an apparatus and a method using alternating magnetic fields for heating compositions comprising magnetically heatable entities that have been targeted at or near the blood-brain barrier.

### BACKGROUND OF THE INVENTION

Brain tumours are extremely lethal and incredibly invasive and therefore, intervening with complex surgery is a top priority in most medical cases. Despite many efforts, drug delivery to the brain remains a challenge mainly because the blood-brain barrier, which consists of are tightly interconnected endothelial cells that cover all the interior of the cerebral vessel walls, is reputed to be insurmountable for most therapeutic molecules. In fact, nearly 98% of new drugs used in the Central Nervous System (CNS) to combat brain cancer and other chronic diseases cannot enter the brain following systemic administration. On the other hand, systemic administration of toxic agents causes the active principles to distribute throughout all the organs. Therefore, while pathological regions are treated, they also promote side effects in healthy organs.

The extremely selective permeability of blood-brain barrier and high cytotoxicity of anticancer drugs reinforce the importance of non-invasive targeted drug delivery for brain tumours and other chronic brain related disorders. Previously, successful local delivery and tracking of therapeutic agents encapsulated in miniaturized magnetic carriers in the liver of a living animal by the gradient field of a modified Magnet has been demonstrated Resonance Imaging (MRI) scanner (P. Pouponneau, J.-C. Leroux, G. Soulez, L. Gaboury, and S. Martel, “Co-encapsulation of magnetic nanoparticles and doxorubicin into biodegradable microcarriers for deep tissue targeting by vascular MRI navigation,” Biomaterials, vol. In Press, Corrected Proof). The proposed carriers consist of therapeutic molecules and aggregates of Magnetic Nanoparticles (Magnetically heatable entities) with relatively high magnetization saturation embedded inside a biocompatible and biodegradable polymer, which serves as a transport mediator in the vasculature. This encapsulation also functions as a protective shield and prevents cells from further exposure to toxic drugs during the carriers' commute to a target area. It will be understood that therapeutic elements comprise chemotherapeutic agents for treating cancer.

Magnetic Resonance Navigation (MRN) relies on Magnetic Nanoparticles (such as magnetically heatable and magneto-responsive entities) embedded into microcarriers or compositions to allow the induction of a directional propelling force by 3D magnetic gradients. These magnetic gradients are superposed on a sufficiently high homogeneous magnetic field (e.g. the Bₒ field of an MRI scanner) to achieve maximum propelling force through magnetization saturation of the magneto-responsive entities. As previously demonstrated by Applicant's group, such a technique was successful at maintaining micro-carriers along a planned trajectory in the blood vessels based on tracking information gathered using Magnetic Resonance Imaging (MRI) sequences from artefacts caused by the same magneto-responsive entities.

Among various known methods of hyperthermia, whole body, microwave and radiofrequency hyperthermia are most commonly used to disrupt the blood-brain barrier. In these techniques an entire region of the brain including neurons, astrocytes, vessel wall cells, and other glial cells are equally heated. In fact, this may be the reason for many undesirable acute side effects with hyperthermic disruption of the blood-brain barrier by these techniques.

Due to the drawbacks of the prior art, it was highly desirable to develop new highly specific methods and apparatuses for delivering drugs to the brain, even though the drugs do not typically cross the blood-brain barrier. The methods and apparatuses would use magnetically heatable entities to generate a targeted and localized heat source in order to permeablize the blood-brain barrier, allowing systemically (intravascular) administered drugs to be targeted to the brain, thereby avoiding the major side effects observed with other non-specific prior art methods.

### SUMMARY OF THE INVENTION

It has been discovered that compositions comprising magnetically heatable entities (MHEs), therapeutic agents and optional carriers such as hydrogels can be piloted from an injection point in a blood vessel to a specific location of the blood-brain barrier (BBB) using for example, a magnetic resonance imaging (MRI) device for propelling, steering and tracking of MHEs. Once the MHEs have reached their target location at or near the desired blood vessel of the BBB, an alternating magnetic field causes the MHEs to controllably heat up, thereby reversibly increasing the permeability of the BBB and allowing the therapeutic (or cytotoxic) agent to enter brain tissue. The MRI device can also be used for indirect determination of local temperatures at a target location for hyperthermia.

In some aspects of the present invention, there is provided a method of delivering an agent to brain tissue comprising providing the agent within a blood vessel with magnetically heatable entities, then targeting the magnetically heatable entities at or near a blood vessel of a blood-brain barrier, causing the magnetically heatable entities to generate heat using an alternating magnetic field, the heat for increasing a permeability of the blood-brain barrier; allowing at least a portion of the agent to cross the blood-brain barrier from the blood vessel to the brain tissue.

In some embodiments, at least partially saturating magnetic fields generated using a magnetic resonance imaging device are used for propelling the magnetically heatable entities using the gradient coils of the imaging device.

In other embodiments, the magnetically heatable entities are magnetotactic bacteria and the targeting the magnetotactic bacteria further comprises using magnetic fields for one or any combination of for steering and aggregating the bacteria. The magnetic fields can be generated using one of a 3D Maxwell coil configuration and a 3D Helmholtz coil configuration.

In yet other embodiments, a level of the heat generated at the blood-brain barrier by the magnetically heatable entities is adjusted to achieve a desired permeability of the blood-brain barrier. In such embodiments a magnetic resonance imaging device comprises a temperature determinator for determining a temperature of the brain tissue.

In still other embodiments, the agent comprises one or more of a therapeutic element, a diagnostic element and a prophylactic element. In such embodiments, the agents can be encapsulated with the magnetically heatable entities in a thermo-sensitive hydrogel carrier, such as a hydrogel comprising poly(N-isopropylacrylamide). Alternatively, the magnetically heatable entities can be antibody-based or chemically cross-linked to the agent.

In some aspects of the present invention, there is provided an apparatus for locally delivering heat to blood-brain barrier tissue for delivering an agent to brain tissue comprising an alternating magnetic field source for heating magnetically heatable entities in a blood vessel at or near and blood-brain barrier to allow passage of the agent across the blood-brain barrier.

In some embodiments, the apparatus further comprises gradient coils such as those in a magnetic resonance imaging device for creating a magnetic field for propelling the magnetically heatable entities to a blood vessel of a blood-brain barrier. In such an embodiment, the imaging device can be exploited for determining a location of the magnetically heatable entities inside the body of a subject/patient.

In some embodiments, the apparatus further comprises a controller configured to send output to the alternating magnetic field source for controlling a level of heat generated by the magnetically heatable entities. The controller can also be configured to receive input from the imaging device concerning a location of the magnetically heatable entities and to send output to the gradient coils for controlling the magnetic field for controlling a locating of the magnetically heatable entities. The controller can also be configured to control the magnetic field source to adjust a level of heat as a function of a desired permeability of the blood-brain barrier. The controller can also be configured to receive

It other embodiments of the present invention, there is provided a system for heating a blood-brain barrier to deliver an agent to brain tissue comprising magnetically heatable entities to be delivered to the blood-brain barrier and an apparatus for heating the magnetically heatable entities when they are at or near the blood-brain barrier.

In some embodiments, the magnetically heatable entities and the agent are encapsulated in a common carrier such as a hydrogel. The hydrogel can comprise poly(N-isopropylacrylamide).

In some embodiments, the magnetically heatable entities have a diameter between 10 nm and 20 nm while in other embodiments, the magnetically heatable entities comprise ferromagnetic particles such as ferric oxide (Fe3O4).

### BRIEF DESCRIPTION OF THE DRAWINGS

The invention will be better understood by way of the following detailed description of embodiments of the invention with reference to the appended drawings, in which:

FIG. 1 shows the experimental schematics for Part i of the study

FIG. 2 shows the elevation of brain temperature as a function of distance from external heating device

FIG. 3 shows the extracted brain of mouse #1 from Group I with Evans Blue dye near the heating point.

FIG. 4 shows brain thermal mapping.

FIG. 5 shows examples of hydrogels for delivery to the brain.

FIG. 6 shows a schematic representation of magnetically heatable entities inside a coil.

FIG. 7 shows the A/C magnetic field induced temperature increase caused by magnetically heatable entities.

FIG. 8 shows a schematic representation of an embodiment of a system for delivering an agent to the brain.

FIG. 9 shows a highly schematic representation of a hydrogel targeted to the blood-brain barrier and ready to be heated by an alternating magnetic field.

FIG. 10 shows the temperature profiles for various MHEs as a function of time upon exposure to an alternating magnetic field.

FIG. 11 shows top and bottom views of mice brains after injection of MHEs (or not) and exposure (or not) to an alternating magnetic field.

### DETAILED DESCRIPTION

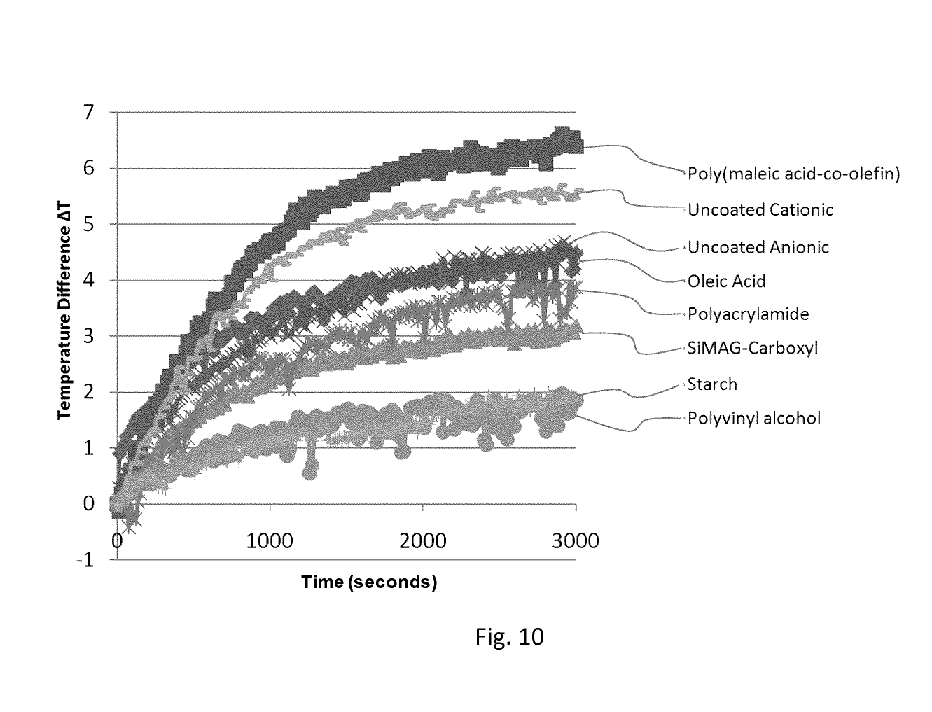

Besides propulsion and tracking, magneto-responsive entities can be synthesized with characteristics that allow for the diffusion of therapeutic cargo carried by these MR-navigable carriers through the blood-brain barrier using localized hyperthermia without compromising their magnetic navigation capabilities. When the magneto-responsive entities also have the property of being heatable using an alternating magnetic field, they will also be called magnetically heatable entities (MHEs). Localized hyperthermia induced by an alternating magnetic field (AC field) is demonstrated for the purpose of transient controlled disruption of the blood-brain barrier and hence local delivery of therapeutic agents into the brain. Initially, an external heating apparatus was used to impose a regional heat shock on the skull of a living mouse model. The effect of heat on the permeability of the blood-brain barrier was assessed using histological observation and tissue staining by Evans blue dye. Results show direct correlation between hyperthermia and blood-brain barrier leakage as well as its recovery from thermal damage. Further experiments have demonstrated that intravascularly injected of MHEs can be targeted to the brain and heated up and to disturb the blood-brain barrier (see FIG. 9). Therefore, in addition to on-command propulsion and remote tracking, the proposed navigable agents can control opening of the blood-brain barrier by hyperthermia and selective brain drug delivery.

Inside brain microvasculature, heat may thermally disrupt an intact blood-brain barrier thereby creating a transient opening for the therapeutic agents to cross into the brain tissue. In fact, it has long been recognized that hyperthermia, otherwise known as elevation of body temperature, can lead to intense cellular stress and cause temporal disruption of the blood-brain barrier as well as death of cancer cells by enhancing cell sensitivity and vulnerability towards more established forms of cancer therapy, such as radiation and chemotherapy.

Here, integration of the MHEs with MRI-based propulsion and tracking technique allow for interventions in the vasculature requiring local hyperthermia. Results provided herein show that there may be a direct relationship between the elevated brain tissue temperature and the extent of the penetration of the desired drug molecules across the blood-brain barrier. This implies that by controlling the amount of heat and exposure time, Applicants can adjust the blood-brain barrier opening for various molecular dimensions. Finally the recovery of the blood-brain barrier from thermal damage is examined.

All capillaries in the mammalian body including humans are composed of endothelial cells. In the circumventricular organs, most of the capillaries are fenestrated to allow for rapid exchange of molecules such as the therapeutic agents between blood vessels and surrounding tissue. In the rest of the brain however, very complex inter-endothelial tight junctions interconnect the endothelial cells. The tight junctions seal the interstitial space and form a diffusion barrier that markedly controls the flow of molecules across the epithelium. In addition to the tight junctions, pericytes with smooth muscle-like properties constitute the blood-brain barrier. Only small electrically neutral lipid-soluble compounds with a molecular mass of less than about 400-500 Daltons (Da), or those small electrically neutral lipid-soluble compounds with low air-water partition coefficients and an average cross-sectional area of 50 Å², are able to diffuse passively through the blood-brain barrier. As mentioned before, this restrains admission of a considerable portion of pharmaceutical agents into the brain. For instance, currently many large drug molecules such as peptides, recombinant proteins, monoclonal antibodies, antisense and non-viral gene medicines are ineffective for the brain.

One of the main functions of the blood-brain barrier is to keep the neurotransmitters and agents that act in the CNS separate from the peripheral tissues and blood, so that similar agents can be used in the two systems without “cross-talk”. Also, because of the blood-brain barrier's large surface area (180 cm² per gram brain tissue) and the short diffusion distance between neurons and capillaries (8-20 μm), the extent to which a molecule enters the brain is determined only by the permeability characteristics of the blood-brain barrier and that has a predominant role in regulating the brain microenvironment. That is why circumventing the blood-brain barrier is a priority for any drug delivery mechanism to the region of the brain. Successful crossing of this barrier will have a profound effect on the treatment of many brain related disorders.

In modern oncology, hyperthermia generally refers to heating of organs or tissues in various ways to temperatures between 40° C. and 45° C., at which point it causes moderate and reversible cellular inactivation. In this regard, induction of magnetically heatable entities by an AC field is investigated for elevation of tissue temperature. Magnetically heatable entities can act as very small heat sources once placed in an AC field, regardless of their depth inside a biological entity. On the contrary, techniques such as RF, microwave and High Intensity Focused Ultrasound (HIFU), are not able to accurately target desired deep-seated tissues.

During local hyperthermia to temperatures near 42° C. in the region of the brain (ΔT=5° C.), morphological changes of individual endothelial cells in the monolayer lining of the micro-vessels begin to cause the tight junctions between adjacent endothelial cells to loosen, therefore allowing transport of large molecules through intercellular pathway. The blood-brain barrier has the capability to restore functionality after brief hyperthermic disruption. The rate of this restoration however, depends on the amount of heat and the exposure time And this is why a critical aspect of the present invention resides in the control local temperatures induced by the MHEs on the blood-brain barrier.

In the proposed hyperthermic disruption of the blood-brain barrier by induction of magnetically heatable entities inside an AC field, heat is exclusively dissipated to the ambient vessel wall cells by thermal conduction. Consequently, only the monolayer lining of the vessel walls and the endothelial cells are directly affected by the thermal stress.

The Specific Absorption Rate (SAR) or heat generated by the magnetically heatable entities is mainly caused by three major mechanisms; hysteresis loss, Néel, and Brownian relaxations. Particles' physical properties as well as magnitude and frequency of the applied AC field determine the relative strength of each of these mechanisms. SAR is proportional to the time rate of change of temperature of a magnetic material and is given by the following formula:

$$SAR = \frac{{cV}_{s}}{m} \frac{d T}{d t} \qquad ( 1 )$$

In (1), c is the specific heat capacity of the sample (J≠I⁻¹·K⁻¹), m is the mass of the magnetic particles (kg), Vₛ is the total volume (m³) and dT/dT expressed in ° K·s⁻¹, is the temperature increment which is experimentally derived from the linear regression of the initial data points obtained from the time varying temperature curve. In the steady state, the difference in temperature ΔT is given by (2) where C is the concentration of the magnetically heatable entities (mass of the particles per tissue volume) and A represents the heat conductivity of a tissue volume with a radius R.

$$Δ T = SAR \frac{{CR}^{2}}{3 λ} \qquad ( 2 )$$

From (2) it is evident that higher concentration of magnetically heatable entities per unit volume of the tissue leads to higher ΔT. In reality, for most therapeutic applications, the relatively poor energy transfer efficiency of the magnetically heatable entities, i.e. poor SAR, introduces a great obstacle that hinders full functionality or demands large administration of the magnetically heatable entities at the biological target location leading to an increase in possible side effects. That explains why magnetically heatable entities with the highest possible SAR are highly desirable.

In contrast to other forms of magnetization, superparamagnetism can prevent formation of nanoparticle clusters in the biological entity. That is why ultra-small magnetite or superparamagnetic iron oxide (magnetite: Fe₃O₄) nanoparticles have been given special attention for hyperthermia. These particles are commercially available and their physical properties are vastly studied. In addition, magnetite nanoparticles have shown great biocompatibility, biodegradability and low toxicity. The SAR value of these particles varies with respect to particle diameter and magnetic properties of the AC field. Applicant's previous studies with superparamagnetic magnetite nanoparticles have shown promising results with regards to hyperthermia (S. N. Tabatabaei, J. Lapointe, and S. Martel, “Shrinkable Hydrogel-Based Magnetic Compositions for Interventions in the Vascular Network,” Advanced Robotics, vol. 25, pp. 1049-1067, 2011). Table 1 summarizes some of the key parameters required to elevate the temperature of the brain tissue from 37° C. to 42° C. using commercially available particles. These parameters are induced from numerous in-vitro experiments and simulations presented in Applicant's previous studies (S. N. Tabatabaei, “Evaluation of hyperthermia using magnetic nanoparticles and alternating magnetic field,” Master, Institute of Biomedical Engineering, University of Montreal, Montreal, 2010). In the same table, magnetically heatable entities with much higher SAR but not yet commercially available are also reported (J.-H. Lee, J.-t. Jang, J.-s. Choi, S. H. Moon, S.-h. Noh, J.-w. Kim, J.-G. Kim, I.-S. Kim, K. I. Park, and J. Cheon, “Exchange-coupled magnetic nanoparticles for efficient heat induction,” Nat Nano, vol. 6, pp. 418-422, 2011).

Below a specific diameter, some magnetically heatable entities become superparamagnetic. For instance, for iron oxide (Fe3O4) magnetically heatable entities of less than 20 nm in diameter, the orientation of the magnetic moment continuously changes due to thermal agitation. By applying an external magnetic field to these magnetically heatable entities, the energy from the field drives the magnetic moments to rotate and aligns them with the magnetic field direction by overcoming the thermal energy barrier. However, once the external magnetic field is removed, magnetic moments do not relax immediately, but rather take a certain time to return to their original random orientation. This is known as the Néel relaxation mechanism. During the relaxation period, the magnetic field energy is released from the magnetically heatable entities in the form of heat. As such, magnetically heatable entities injected in the human body can serve as nano-sized heat sources once the body is placed inside an AC magnetic field in which the external magnetic field amplitude switches intensity at a given frequency. The intensity of the heat that is generated by the AC magnetic field depends mainly on the size, distribution, concentration and chemical composition of the magnetically heatable entities.

For maximum penetration of the electromagnetic energy, it is necessary to select a frequency in accordance with the targeted depth. This can be explained by the fact that an electromagnetic wave passing through the human body would reduce in intensity. To penetrate electromagnetic energy approximately 10 cm inside the tissue unscattered and unabsorbed, frequencies in range of 100 kHz have been suggested but any frequency able to penetrate biological tissue would effective, to various degrees.

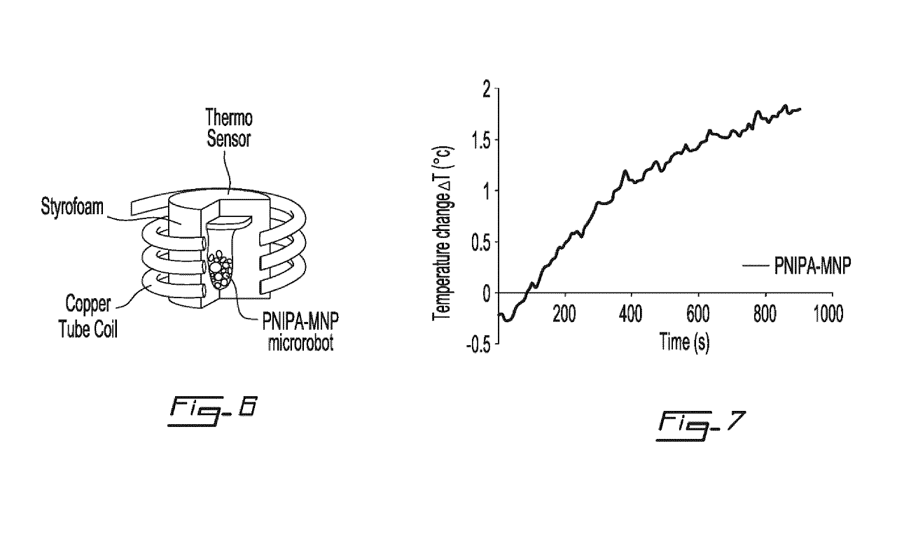

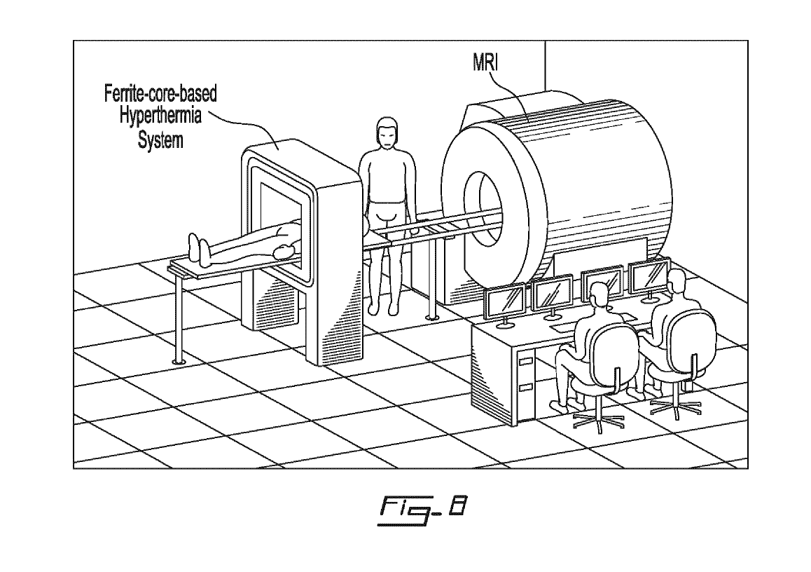

A systematic hyperthermic actuation mechanism for the compositions has been realized by encapsulating magnetically heatable entities in thermo-sensitive PNIPA hydrogels. The sponge-like property of the PNIPA-magneto-responsive entity compositions allows them to release their contained liquid once sufficient heat is induced. In the present experiment, such heat came from hyperthermia induced by the magnetically heatable entities embedded in the compositions via the AC magnetic field as described earlier using a setup configuration seen in FIG. 6. Results from hyperthermia of the compositions inside an AC magnetic field of 4 kA/m at 160 kHz are depicted in FIG. 7. The temperature change ΔT was approximately 2° C. for a treatment period of 900 s. This time frame is subject to increase or decrease in harmony with the decrease or increase of the AC magnetic field amplitude and/or frequency, respectively. In addition, the magnetic properties of the magnetically heatable entities used in the compositions would also have an impact on the time frame of the final temperature. In other words, the present compositions were able to release water molecules as much as 25% of their initial volume once their temperature increased from 33.5 to 35.5° C.

As mentioned above, ΔT can reach higher values once the compositions are equipped with optimized magnetically heatable entities. For ΔT=5.5° C. In the region of the AC magnetic field, Applicants assume uniformity of the field for many reasons. Chief among those is due to the small distribution size of the magnetically heatable entities compared to the coil dimensions. Also, the compositions were positioned in the center of the AC magnetic field where the field was most uniform. The lower critical solution temperature (LCST) of the compositions can be adjusted slightly above human body temperature. Hence, by tuning the LCST of the PNIPA-MNP (magnetically heatable entity) drug-carrying compositions to 39° C., the AC magnetic field of 4 kA/m at 160 kHz would be able to provide sufficient heat to trigger a drug release sequence inside the vasculature near the blood-brain barrier.

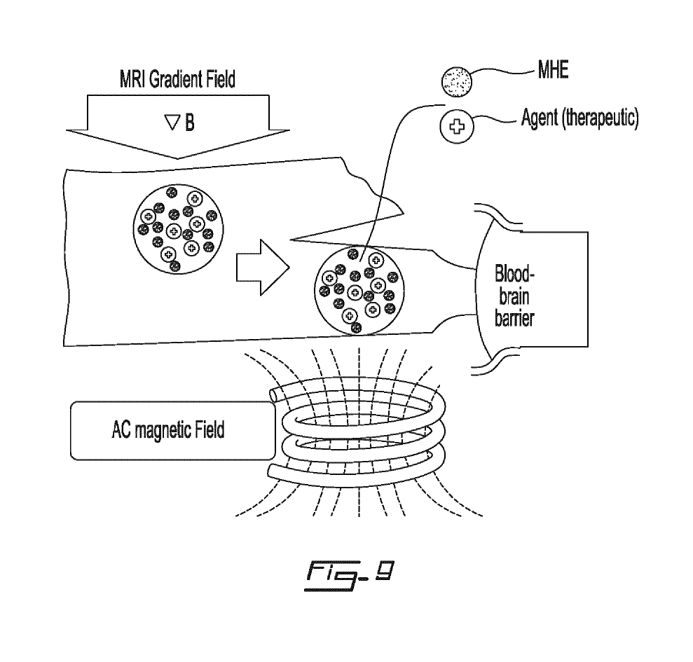

The AC magnetic field for inducing hyperthermia of the compositions can be generated via several different coil designs. Nevertheless, the technical and medical requirements such as the precision of the magnetic field strength, frequency and uniformity, as well as safety and the clinical quality of the treatment procedure, limit these designs for human-scale configuration. As seen in FIG. 8, the simplest approach is a cylindrical coil in the middle of which a patient is comfortably placed, where the AC magnetic field is most uniform. FIG. 8 depicts a human-scale system in which propulsion, tracking and actuation of the compositions in the vascular network is possible. After injection, the patient is placed inside the MRI for magnetic resonance tracking and steering of the compositions. Once the compositions have reached the desired location, they become stationary due to their size, and, as seen in highly schematic FIG. 8, embolized at the far ends of small blood vessels near the blood-brain barrier area. At this time, the patient is rolled outside of the MRI and placed inside the hyperthermia system where the AC magnetic field finalizes the drug-release mechanism sequence. A great advantage of this technique for the patient, field doctor and technicians is that in the case that repetition of the procedure is recommended, the patient is easily rolled back into the MRI and compositions can be re-injected for further drug delivery.

TABLE 1
Parameters required to elevate tissue temperature
5° C. by hyperthermia of magnetite

| | Commercial | Lee, J H et al. |
|---|---|---|
| Composition | Fe₃O₄ | CoFe₂O₄ |
| Diameter (nm) | 10 nm | 9 nm |
| Magnetism | Superpara | Superpara |
| Coating | PMO | MnFe₂O₄ |
| AC field amplitude (kA/m) | 4.5 | 37.3 |
| AC field frequency (kHz) | 160 | 500 |
| SAR (W/g) | 61.02 | ~3000 |

The main difficulty of localized hyperthermic disruption of the blood-brain barrier by induction of magnetically heatable entities is transportation of the magnetically heatable entities and therapeutic agents through the vasculature to a desired area of the brain. First, the carrier must have the ability to geometrically fit into the target microvasculature. Second, the carrier must allow for maximum magneto-responsive entity concentration at the target area in order to reach sufficient thermal levels. It is also important to consider factors such as immunological reactions, excessive toxicity, premature degradation and fast excretion of the carrier by blood enzymes, or unexpected capture by non-targeted tissues that may affect the carrier behavior. Compositions such as polyglycolic acid (PGA), poly(lactic-co-glycolic acid) (PLGA), and various environment-sensitive hydrogels are some of the well-known biomaterials for this purpose.

In some embodiments, for the purpose of increasing the efficiency of targeting MHEs to the blood-brain barrier, the MHEs can be coated with specific epitopes that are recognized by and/or interact with cell surface moieties found of endothelial cells of the blood-brain barrier. Conversely, the MHEs (or hydrogel compositions comprising MHEs) can be coated with antibodies that recognize specific cell surface antigens found on endothelial cells of the blood vessels of the blood-brain barrier. Using one of the techniques allows to increase targeting of the MHEs to the blood brain barrier

Magnetic nanoparticles experience a thrust force when exposed to a gradient field, such as in an MRI machine. As seen in equation (3), this magnetic force, \vec{F}_{magnetic} (N) is directly proportional to the volume of the magnetically heatable entities, V_{ferro} (m³) and their magnetization properties, \vec{M} (A·m⁻¹) as well as the gradient of the magnetic field, ∇\vec{B} (T).

\vec{F}_{magnetic}=V_{ferro}·M·∇\vec{B} (3)

Magnetic Drug Targeting (MDT) techniques known in the art use this same principle to concentrate therapeutic drugs adsorbed, entrapped or covalently linked to aggregates of magnetically heatable entities at a superficial target location following a local intravenous injection. The main difficulty of this technique however, is that it lacks the ability to target deep tissues. Therefore, instead of a conventional approach most often based on an external magnet, an improved alternative method based on the three dimensional gradient magnetic field of the MRI was developed by the Applicant. In this technique, due to the large main magnetic field of the MRI, aggregates of the magnetically heatable entities become magnetically saturated once inside the relatively high homogeneous field of a clinical MRI scanner and therefore, relatively small shifts in the gradient field can steer them towards a target anywhere in the tissue. Furthermore, the aggregates of the magnetically heatable entities create a magnetic distortion on the images acquired by MRI sequences. Therefore, the same MRI platform is able to track the aggregates in real-time and confirm their presence at the target.

In order to manoeuvre in the microvasculature, and to avoid fast degradation of the magnetically heatable entities in the blood circulation system, as well as preventing them from dissociation, which greatly limits the thrust force and distortion for tracking purposes, the magnetically heatable entities along with the therapeutic agents can, in some embodiments, be encapsulated in biocompatible micrometer size carriers. Previously, Applicant was able to synthesize such complex microcarriers and to use them to target a specific region of the liver of a living rabbit (Pouponneau et al., 2012) prior to the successful release of the therapeutic agent.

Various studies evaluated the required amount of thermal exposure and duration for temporal disruption of the blood-brain barrier based on imposition of thermal stress on the tight junctions (J. Lin and M. Lin, “Microwave hyperthermia-induced blood-brain barrier alterations,” Radiation Research, vol. 89, pp. 77-87, 1982; and E. A. Kiyatkin and H. S. Sharma, “Permeability of the blood-brain barrier depends on brain temperature,” Neuroscience, vol. 161, pp. 926-939, 2009). According to the findings of these studies, the ideal temperature for transient disruption of the blood-brain barrier falls in the range of 42° C.-44° C. for a period of 30 minutes.

Staining of the blood-brain barrier is a traditional method for evaluating blood-brain barrier leakage. Evans Blue dye, an exogenous tracer, is used to assess the integrity of the blood-brain barrier following a hyperthermic disruption. The dye molecules are able to easily diffuse through the fenestrated endothelial cells of all capillaries except those of the brain due to a functional blood-brain barrier. However, once the blood-brain barrier is compromised, Evans blue enters the brain and it fluoresces with excitation peaks at 470 and 540 nm and an emission peak at 680 nm. Histological staining techniques can therefore reflect the extent of blood-brain barrier leakage by studying the intensity of Evans blue dye in the brain.

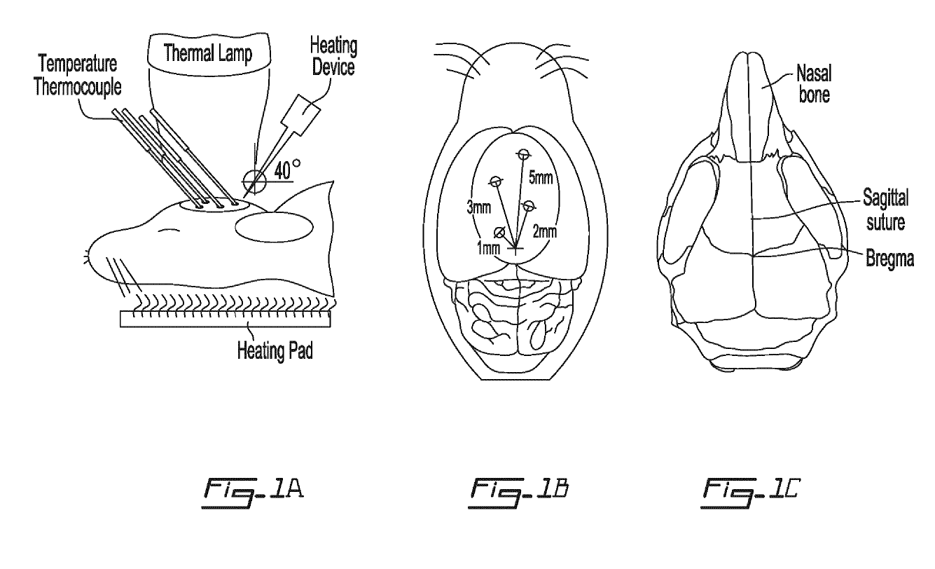

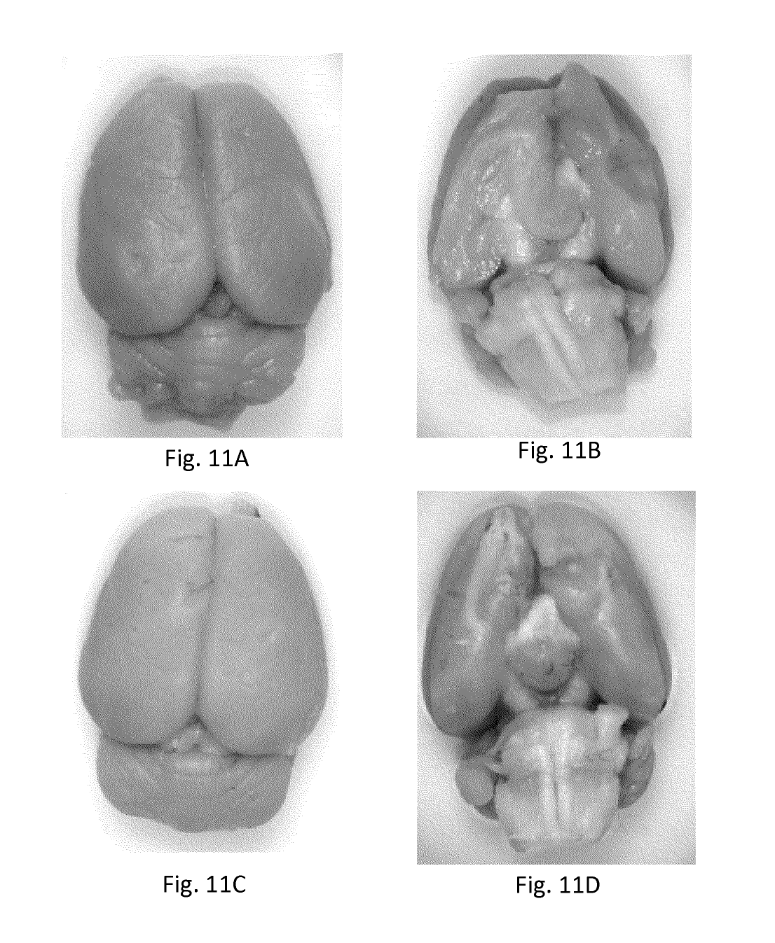

FIG. 1A shows the experimental schematics for Part i) of the experimental procedure below, while FIG. 1B shows a dorsal view of the brain. The cross in the middle represents the position of the heating point. FIG. 10 shows that heating was done over the skull near Bregma.

### Experimental Procedures

In the primary steps of this study, distribution of heat from an external heating device on the brain of living mice as well as feasibility studies in regards to hyperthermic disruption of the blood-brain barrier were assessed. For this purpose, a two-phase in-vivo experiment was executed:

Phase i. Five mice were separately anesthetised by intravenous injection of 40 mg/kg body weight of pentobarbital. Quickly thereafter, each animal was positioned on a stereotaxic frame and the head of the animal was secured in place. Then, by removing the skin, the surface of the skull of the animal was exposed. At this point, four small holes (˜1 mm in diameter) were drilled into the skull at precise locations shown in FIG. 1A. Next, fibre optic thermocouples were placed inside the holes. An external heating device was used to focally elevate the temperature of a small region of the brain near Bregma (see FIG. 10) at a 40° angle for a duration of 30 minutes. While the thermocouples recorded changes in temperature at specific distances (1, 2, 3, 5 mm respectively) away from the heating point (see FIG. 1B).

During the procedure, a rectal thermometer monitored the internal body temperature of the animal. Since the body temperature drops rapidly during anaesthetic state, each animal was placed on a thermal pad. In addition, a heating lamp was placed 5 cm above the skull to keep the brain temperature at 37° C. during the experiment. As a consequence, the body temperature was always kept between 36.5° C. and 37° C.

The purpose of this experiment was to examine the thermal distribution in brain tissue. This resulted in a thermal map of the tissue represented in Section IV. The environment of the experimental suite was kept thermally neutral during all experiments.

Phase ii. The purpose of this part of the experiment was to examine the feasibility of hyperthermic disruption of the blood-brain barrier as well as its recovery period from thermal damage using Evans blue staining technique. Here, nine mice, each 6-8 weeks of age, were randomly divided into three identical groups. In contrast to the previous part, no holes were drilled into the exposed surface of the skull of these animals. In order to correlate temperature patterns with stains left from the Evans blue dye, the heating parameters for all groups were kept the same as was described in the first part of this experiment.

Group I All mice in this group were intravenously injected with 40 mg/kg of body weight of pentobarbital and 4 ml/kg body weight 2% Evans blue dye. As in Part i, and quickly after anaesthesia each animal was positioned on a stereotaxic frame where a thermal pad and the lamp kept the body temperature steady at near 37° C. Just as before, the heating device was also placed at a 40° angle near and above Bregma and the exposure time was set to 30 minutes. All animals in this group were sacrificed one hour after injection of the dye. The animals' brains were extracted and immersed in isopentane and kept on dry ice for further analysis.

Group II. To examine blood-brain barrier's ability to recover following hyperthermic disruption, all mice belonging to this group were prepared the same way as done in Group I except that the dye was injected 2 hours after 30 minutes of thermal treatment had ended. Exactly one hour after injection of the dye, the animals were sacrificed and their brains were removed, immersed in isopentane and kept on dry ice for further study.

Group III. All mice in this group served as controls. There was no staining of the Evans blue found on the brain tissue of the mice from this group.

Data Analysis. Extracted brains were embedded in Optimal Cutting Compound where 50-micron coronal slices were made at −20° C. in a cryostat. Results are shown in the next Section.

Results from the first part of the experiment are shown in

. In this figure, recorded temperatures from 1 mm and 2 mm distances away from the heating device quickly raised once heating started and rapidly plateaued at approximately 44.3° C. and 39.4° C. respectively. Also, at the distances of 3 mm and 5 mm away from the heating device, temperatures reached 37.8° C. and 37.6° C. respectively.

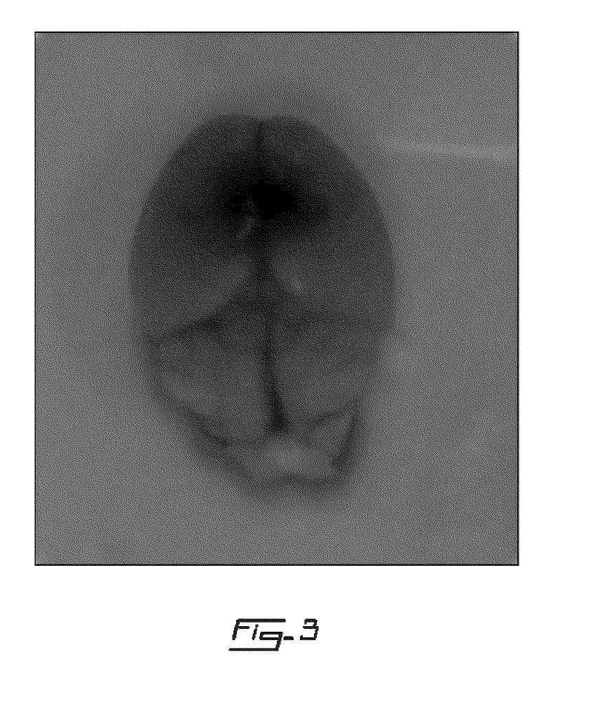

In phase 2, Evans blue dye was expected to have distributed throughout the entire body except the brain where it is forbidden entry (prior to the hyperthermic disruption of the blood-brain barrier). Following hyperthermia, the extracted brains from the animals of Group I revealed that hyperthermia could indeed disrupt the blood-brain barrier and allow entry of a large and heavy molecule such as Evans blue into the brain tissue illustrates what the extracted brain from a mouse in this group resembles. As it is seen in FIG. 3, the integrity of the blood-brain barrier where heat was applied was severely compromised leading to staining of that region of the brain. FIG. 3 shows the appearance of the Evans blue dye near and around the heating point (Group I Animal #1).

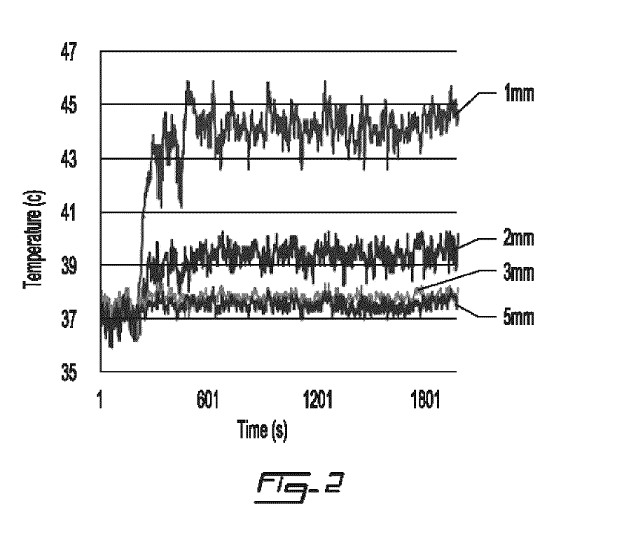

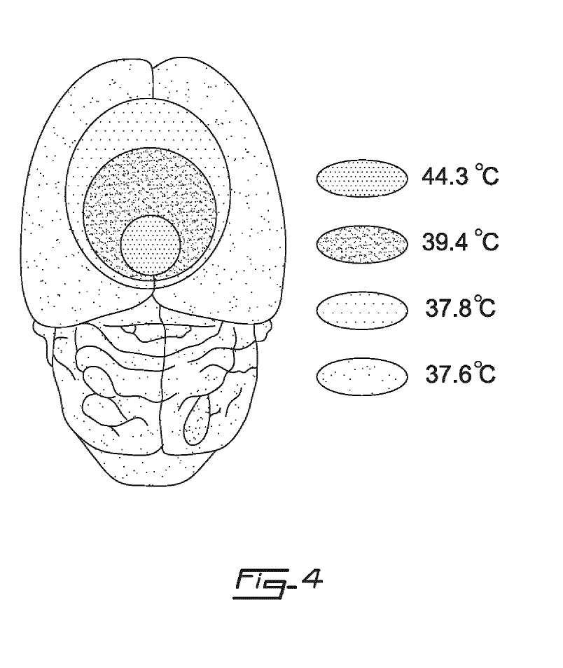

FIG. 4 was generated based on a correlation with the temperature data from and FIG. 1b. The temperature curves presented in indicate conduction of heat in the brain tissue regardless of the method with which heat has been produced. As mentioned before, magnetically heatable entities are also able to create such thermal energy by relaxation processes. Thus, in the presence of micro-robots near the Bregma, distribution of heat generated by excitation of the embedded magnetically heatable entities inside an AC field would be similar to that of FIG. 2. Results from the histological examination of the extracted brains are tabulated and presented in table 2. As seen, all animals from the first group except one (#3) were affected by hyperthermia where Evans blue left a visible stain on the brain tissue around Bregma. It is believed that technical problems caused the anomaly for animal #3. Animals from the second group that received a 2-hour recovery period, showed substantially lower leakage area compared to the animals in Group I.

TABLE 2
Histological examination of the blood-brain
barrier leakage of Evans blue dye.

| | | Heating point | Diameter of |
|---|---|---|---|
| | | position away | BBB leakage of |
| Phase II | Animal # | from Bregma (mm) | Evans blue (mm) |
| Group I | 1 | 1.6 | 4.22 |
| Hyperthermic | 2 | 2.5 | 2.46 |
| Disruption | 3 | 1.66 | 0 |
| Group II | 1 | 2.3 | 0 |
| Recovery from | 2 | 2.4 | 0.62 |
| Hyperthermia | 3 | 2.2 | 1.98 |
| Group III | 1 | N/A | 0 |
| Control | 2 | N/A | 0 |
| | 3 | N/A | 0 |

The location of the leakage of the blood-brain barrier and visible stains were also very close to the heating device where according to

must have reached to temperatures higher than 40° C. Therefore, although the blood-brain barrier seems to have partially or in case of Animal #1 fully recovered from hyperthermic disruption, the recovery period may not have been sufficient for complete rejuvenation of the blood-brain barrier. No evidence of blood-brain barrier leakage for Group III could be found.

Overcoming the blood-brain barrier is an important field of current research that seeks a technique to reach the inside of the brain. To achieve brain localized drug delivery and increased efficacy, therapeutic agents are administered to the brain by means no more invasive than an intravenous injection of microcarriers or compositions consisting of magnetically heatable entities and therapeutic agents capable of remote propulsion and tracking compatible with MRN, and on-command actuation in the brain. The results of the experiments presented herein indicate that temperatures of 38° C. and higher for an exposure time of 30 minutes are required for effective hyperthermic disruption of the blood-brain barrier for crossing of large and heavy drug molecules. This crossing however is governed by the change of thermal energy or ΔT, which according to equation (2), is directly depended to the value of SAR. Therefore, controlling SAR leads to controlling the level of blood-brain barrier leakage. For hyperthermia by induction of magnetically heatable entities inside an AC field, as intended herein, the SAR mainly depends on the field frequency and amplitude. Varying these parameters therefore, results in adjusting the blood-brain barrier leakage to Applicant's favour. Thus, Applicants shave shown that this technique not only can be highly localized, it also provides advanced control over the opening of the blood-brain barrier into brain tissue. Because hyperthermia can be dangerous and lead to permanent damage of blood-brain barrier and/or brain tissue, it is essential to have tight control over the temperatures generated by the MHEs. When targeting MHEs to specific locations of the blood-brain barrier using an MRI machine, an indirect determination of temperature can performed in real-time using the MRI with software/calculation specifically configured for this purpose. Obtaining temperature readings from inside brain tissue will most preferably been performed by non-invasive techniques such as the one described above, as also shown in FIG. 8.

It will be understood that brain tissue comprises cells found on the non-vascular side of the blood-brain barrier and is thus composed mainly of neurons and glial cells such as astrocytes, oligodendrocytes and ependymal cells.

The disturbance of the blood-brain barrier at high levels may cause vasogenic edema and energy metabolic failure leading to subsequent structural brain damage. Evidently, the degree of pathophysiological changes in the vascular system of normal brain tissue is dependent on temperature and duration of heating. To minimize local hypo-perfusion and local brain cell death, thermal dosage as well as exposure period should be carefully selected and performed in a controlled environment.

The main goal of modern pharmacology is the delivery of active drug molecules to specific targets. However, nearly 98% of drugs cannot enter the brain following systemic administration. Applicant's group has previously pioneered an MRI-based drug delivery platform referred to as MRN that employs microcarriers or future compositions capable of interventions in the vasculature. Here, Applicants developed micro-entities with hyperthermic capabilities to disrupt the blood-brain barrier and therefore be effective for delivery of therapeutic agents into the brain. This ability comes from the fact that these micro-entities rely on embedded magnetically heatable entities that are excited once placed inside an AC field. This excitation leads to moderate elevation of temperature and thus transient disruption of the blood-brain barrier. In light of this technique, local drug delivery for disorders other than treating brain tumours such as psychiatric, neurological and neurodegenerative disorders as well as any disease requiring delivery of therapeutic agents to the brain will also be feasible.

In an embodiment of the present invention, a controller is provided that receives input from a location of the magnetically heatable entities inside the body of a subject. The controller processes the location information and provides output to gradient coils of an MRI device for piloting the entities to the desired location at or near a blood vessel of the blood-brain barrier. Targeting of the magnetically heatable entities to the desired location can be performed manually by an operator based on the “visually observed” location of the entities but it can also be performed automatically by the controller, programmed for such a purpose. The controller also sends output to the alternating magnetic source to cause the entities to heat up once they have reached the target location at or near the blood-brain barrier.

It will be understood by those skilled in the art that the magnetically heatable entities should be biocompatible with the human body in order to prevent toxicity and/or destruction by immune reaction/rejection. Although some magnetically heatable entities may by “biocompatible” as stand-alone entities in a blood vessel, other entities may not. In such cases, they can be encapsulated in a carrier for transporting the entities to the desired location.

In some embodiments, the carrier can also carry an agent to the desired location. It is understood that it is the agent, and not the magnetically heatable entities, that has therapeutic, diagnostic or prophylactic properties. In some cases, the agent and the magnetically heatable entities are colocalised (but not chemically linked) in a common carrier such as a hydrogel while in other cases, the agent is chemically or physically cross-linked to the magnetically heatable entities. In yet other cases, the magnetically heatable entities are targeted to the blood-brain barrier separately from the agent.

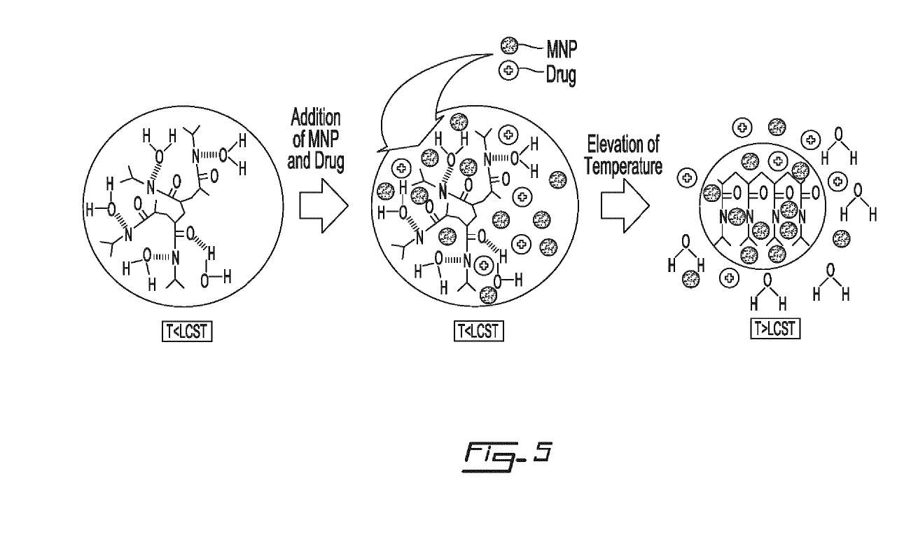

Hydrogels for use as carriers are schematically illustrated in FIG. 5, depicting magnetically heatable entities and drugs (agents) encapsulated in the PNIPA hydrogel polymers at temperatures below the lower critical solution temperature (LOST). The drug and MHE are expelled from the PNIPA hydrogel when the temperature increases above the LOST.

Magnetotactic bacteria of type MC-1 is an example of a biological steerable self-propelled entities (SSPEs) where the flagella bundles are the propulsion (propulsive) system and the chain of membrane-based nanoparticles (crystals) known as magnetosomes embedded in the cell implements such steering system by acting like a miniature magnetic compass needle that can be oriented with a directional magnetic field.

Such magnetotactic bacteria could be used as MHEs if a sufficient quantity/concentration of heat can be generated by the ferromagnetic particles of the bacteria's magnetosomes. If the endogenous magnetosomes of the bacteria are not enough to generate the required amount of heat, the bacteria can be coated with additional ferromagnetic particles. Furthermore, magnetotactic bacteria can be selected/cloned for high magnetosome content and modifications can be made to the genome of the bacteria to increase (or decrease) the activity/transcription of pro-magnetosome (or anti-magnetosome) proteins/genes. Any bacteria having a magnetosome could be used for such a purpose.

In order to prevent deleterious physiological responses from time varying magnetic fields, there are guidelines limiting magnetic field parameters. These guidelines are obtained from scientific observations and epidemiological studies. In some embodiments used for biomedical purposes, the frequency of the electromagnetic field should be higher than 50 kHz to avoid neuromuscular electro-stimulation and lower than 10 MHz for appropriate penetration. Available experimental data shows that the resting human body temperature can be elevated up to 1° C. if it is exposed to an electromagnetic field that produces a whole-body SAR of between 1 and 4 W kg-1. Eddie current loss produced by closed currents induced by alternating magnetic flux in a conductive tissue of sufficient area are responsible for this type of heating. Harmful levels of tissue heating can be produced by exposure of the tissue to fields at higher SAR values. An upper limit of 4.85×108 A m-1 s-1 has been established for the product of field amplitude (H₀) and frequency (f) for a single turn induction coil around the thorax of a normal size patient. For such body exposure, a tissue load threshold of 25 mW ml-1 was recommended. Power density absorbed by the tissue is given by:

$${P}_{tissue} = {π}^{2} \frac{{μ}^{2} \text{?} \text{?}}{2} {f}^{2} {r}^{2} \text{?} ( \text{?} )$$

$$\text{?} \text{indicates text missing or illegible when filed}$$

where μ is the permeability and μ0 is the permeability of free space, σT is the conductivity of the tissue, f stands for frequency, H₀ is the external field strength and r is the distance from the central axis of the body. Using the above equation and the upper limit to the product of the field amplitude and frequency.

Further experiments were performed to evaluate the capacity of various MHEs to generate heat upon exposure to an alternating magnetic field. The experimental setup was similar to that described and shown in FIGS. 6 and 7. FIG. 10 shows the change in temperature as a function of time in an alternating magnetic field for poly(maleic acid-co-olefin), uncoated cationic, uncoated anionic, oleic acid, polyacrylamide, siMAG-carbonyl, starch, polyvinyl alcohol. The results showed that the Poly(maleic acid-co-olefin) MHE had the greatest temperature increase as a function of time at the vast majority of times tested, except in the initial period where Oleic acid showed the fastest response (increase in temperature). After a period of 3000 seconds (50 minutes), the increase in temperature generated by the poly(maleic acid-co-olefin) was about 6.5 degrees Celsius. On the other end, starch and polyvinyl alcohol showed the smallest effect as they were only able to generate an increase of about 2 degrees Celsius over 3000 seconds. Poly(maleic acid-co-olefin) showed an ˜3 degree Celsius increase in temperature after only about 500 seconds (8.33 minutes).

Based on these interesting properties, an initial in vivo experiment was carried out where MHEs were injected into the left common carotid artery of an anaesthetised mouse after which the brain was quickly extracted and placed in an alternating magnetic field. Temperature probes were placed inside various regions of the brain and an almost 2 degree Celsius increase in temperature was observed after only 480 seconds (8 minutes) (not shown). These results show that MHEs can be sufficiently heated up to at least temporarily disrupt the blood-brain barrier.

### In Vivo Experiment

In order to confirm that the initial heating experiments shown in FIGS. 1-4 could be reproduced using magnetically heatable entities according to the present invention, a further set of experiments were performed in which the MHEs were injected into the carotid artery of mice and permeability of the blood brain barrier was evaluated with and without an alternating magnetic field to heat up the MHEs.

The experimental protocol essentially consisted of injecting mice intravenously (IV) with 2 ml 4% Evans Blue (EB) dye. The mice were then anaesthetized using isoflurane (O2 @1 and iso. @ 2%) and a 30 minute diffusion period was observed to allow for proper and complete diffusion of the dye in the animal. MHEs were injected with a syringe through a 2 cm tube inserted into the left common carotid artery and advanced to near the middle cerebral artery (MCA) (its junction at the Willis cycle). 100 microliter of 25% dilution of MHEs in water coated with Poly (maleic acid-co-olefin)—purchased from Chemicell, Germany—was then injected via the tube in the MCA of the animal. The tube was thereafter retracted out of the left common carotid and the anaesthetized animal was then left either outside (normothermia) or inside (hyperthermia) the AC field for 30 min. The alternating current field consisted of a frequency of 154 kHz and an amplitude of 191 amps. Quickly thereafter, cardiac perfusion was performed using 120 ml of warm saline to wash out all blood from the blood vessels of the circulatory system. Mice brains were then extracted and placed into 4% paraformaldehyde (PFA) for further analysis.

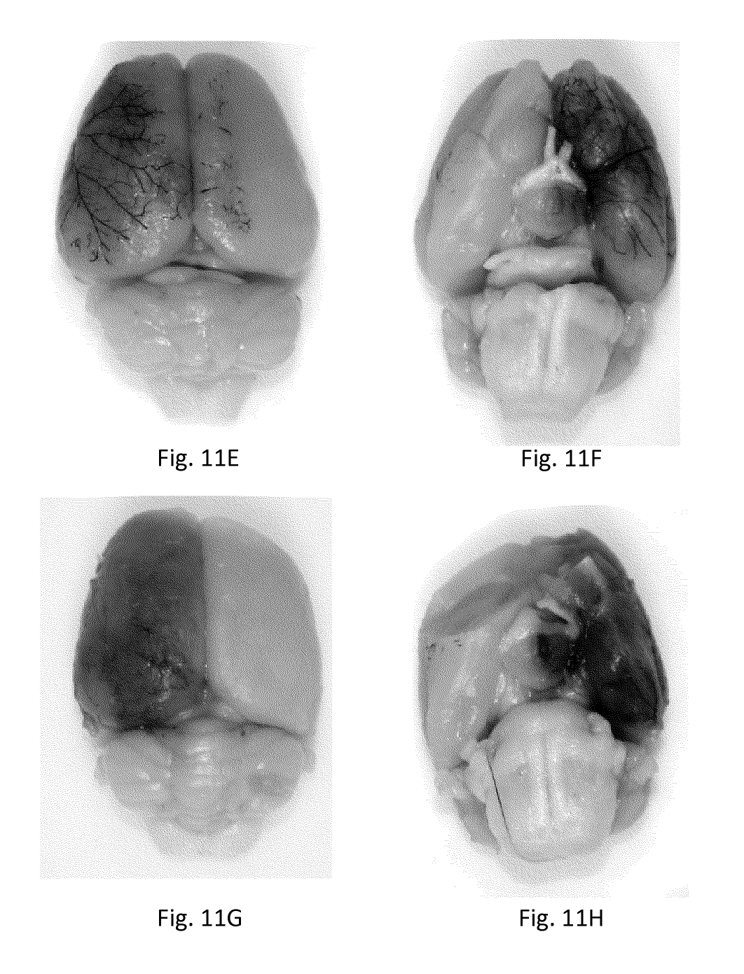

Results of the above experiment are shown in FIG. 11 where FIG. 11A is a top view and FIG. 11B is a bottom view of a mouse brain. This experiment serves as a negative control where, all else being the same, MHEs were not injected and the mice were not placed into the alternating magnetic field device. The results clearly show that no Evans Blue Dye can be observed in the brain tissue of this mouse. It is understood that the blood-brain barrier is in good working order as Evans Blue dye was excluded from brain tissue.

FIG. 11C is a top view and FIG. 11D is a bottom view of a mouse brain and this experiment serves as a negative control for hyperthermia where, all else being the same, MHEs were not injected but where the mice were placed into the alternating magnetic field device for 30 minutes. The results clearly show that no Evans Blue Dye can be observed in the brain tissue of this mouse. It is understood that the blood-brain barrier is in good working order as Evans Blue dye and a 30 minute exposure to an alternating current did not diminish the blood-brain barrier's ability to exclude Evans Blue dye from brain tissue.

FIG. 11E is a top view and FIG. 11F is a bottom view of a mouse brain and this experiment serves as a negative control for hyperthermia (i.e. in normothermia conditions) where, all else being the same, the mice were not placed into the alternating magnetic field device for 30 minutes. In this negative control, MHEs were injected into the carotid artery of the mouse. The results clearly show that some Evans Blue Dye can be observed in the brain of this mouse and that the staining co-localises with blood vessels of the brain. These results suggest that, although Evans Blue dye is observed in the brain, a significant increase in the permeability of the blood-brain barrier cannot be concluded due to the absence of staining in non-vascular brain tissue.

Finally, FIG. 11G is a top view and FIG. 11H is a bottom view of a mouse brain. This experiment demonstrates the ability of MHEs, in the presence of an alternating magnetic field (i.e. in hyperthermia conditions) to cause an extravasation of Evans Blue dye due to the its leakage from blood vessels of the brain to brain tissue.

These results suggest that the magnetically heatable entities were heated up to a sufficient temperature to cause an increase in the permeability of the blood-brain barrier. This increase in permeability is reflected by an diffuse staining of Evan Blue dye throughout the left hemisphere of the brain. Indeed, the normothermia exposed brain of FIG. 11E clearly shows that staining is in, on, or near the blood vessels whereas the hyperthermia exposed brain of FIG. 11G does not specifically stain the blood vessels, rather showing a diffuse staining over the whole hemisphere.

While the invention has been described in connection with specific embodiments thereof, it will be understood that it is capable of further modifications and this application is intended to cover any variations, uses, or adaptations of the invention following, in general, the principles of the invention and including such departures from the present disclosures as come within known or customary practice within the art to which the invention pertains and as may be applied to the essential features herein before set forth, and as follows in the scope of the appended claims.

1. A method of delivering an agent to brain tissue comprising:
providing said agent and magnetically heatable entities within a blood vessel;
targeting said magnetically heatable entities to a blood vessel of a blood-brain barrier;
using an alternating magnetic field for controllably heating said magnetically heatable entities, said heating for reversibly increasing a permeability of said blood-brain barrier;
allowing at least a portion of said agent to cross said blood-brain barrier from said blood vessel to said brain tissue.

2. The method of claim 1, wherein said targeting further comprises using an at least partially saturating magnetic field for propelling said magnetically heatable entities using gradient coils.

3. The method of claim 2, wherein said at least partially saturating magnetic field is generated using a magnetic resonance imaging device.

4. The method of claim 1, wherein said magnetically heatable entities comprise magnetotactic bacteria.

5. The method of claim 4, wherein said targeting said magnetotactic bacteria further comprises using magnetic fields for one or any combination of steering and aggregating said bacteria.

6. The method of claim 4, wherein said magnetic fields are generated using one of a 3D Maxwell and Helmholtz coil configuration or any combination thereof.

7. The method of claim 1, wherein said providing further comprises injecting said magnetically heatable entities and said agent into a blood vessel.

8-9. (canceled)

10. The method of claim 1, wherein said magnetically heatable entities comprise ferromagnetic particles.

11. The method of claim 10, wherein said ferromagnetic particles comprise ferric oxide.

12. The method of claim 1, wherein said agent comprises a therapeutic element.

13. The method of claim 1, wherein said agent comprises one or more of a diagnostic element and a prophylactic element.

14. The method of claim 1, further comprising encapsulating said agent and said magnetically heatable entities in a thermo-sensitive hydrogel.

15. The method of claim 14, wherein said hydrogel comprises poly(N-isopropylacrylamide).

16. The method of claim 1, wherein a linking between said agent and said magnetically heatable entities comprises one of a chemical and an antibody based cross-linking.

17. The method of claim 1, wherein said method further comprises, prior to said heating, moving a patient from a magnetic resonance imaging device to a separate device providing an alternating magnetic field.

18-20. (canceled)

21. The method of claim 1, wherein said controllably heating comprises using a magnetic resonance imaging device for indirectly determining a temperature of said brain tissue.

22. The method of claim 1, wherein said targeting further comprises tracking said magnetically heatable entities using a magnetic resonance imaging device.

23-38. (canceled)

39. A system for heating a blood-brain barrier to deliver an agent comprising:
biocompatible magnetically heatable entities to be delivered to said blood-brain barrier; and
an apparatus comprising an alternating magnetic field source for heating said magnetically heatable entities in a blood vessel at or near and blood-brain barrier to allow passage of said agent across said blood-brain barrier.

40-53. (canceled)

54. A composition of matter comprising magnetically heatable entities and a therapeutic agent for treating brain conditions comprising the steps of:
providing said agent and said magnetically heatable entities within a blood vessel;
targeting said magnetically heatable entities to a blood vessel of a blood-brain barrier;
using an alternating magnetic field for controllably heating said magnetically heatable entities, said heating for increasing a permeability of said blood-brain barrier;
allowing at least a portion of said agent to cross said blood-brain barrier from said blood vessel to said brain tissue.
